(19) United States
(12) Patent Application Publication
(10) Pub. No.: US 2021/0106460 A1
(43) Pub. Date: Apr. 15, 2021

(54) METHOD FOR USING BEAUTY INSTRUMENT WITH MASK

(71) Applicant: Beijing FUNATE Innovation Technology Co., LTD., Beijing (CN)

(72) Inventors: LI FAN, Beijing (CN); LI QIAN, Beijing (CN); YU-QUAN WANG, Beijing (CN)

(21) Appl. No.: 16/708,778

(22) Filed: Dec. 10, 2019

(30) Foreign Application Priority Data
Oct. 15, 2019 (CN) 201910980370.9

(51) Int. Cl. A61F 7/00; A45D 44/00

(52) U.S. Cl. CPC A61F 7/0085; A61F 7/00; A45D 44/002; A61F 2007/0052; A61F 2007/0007; A61F 2007/0015; A61F 2007/0014; A61F 2007/0003

(57) ABSTRACT

A method for using beauty instrument with mask includes steps of: providing a beauty instrument with mask; applying the flexible mask of the beauty instrument with mask on a user's face; and turning on the controller and selecting a function button from the plurality of function buttons on the controller, inputting a current to the at least one heating layer in the flexible mask, and heating the at least one heating layer. The beauty instrument with mask includes a flexible mask and a controller. The controller is electrically coupled with the flexible mask.

### CROSS-REFERENCE TO RELATED APPLICATIONS

The application is also related to copending applications entitled, “BEAUTY INSTRUMENT WITH MASK”, filed **** (Atty. Docket No. US78580).

### FIELD

The subject matter herein generally relates to a method for using beauty instrument with mask.

### BACKGROUND

With the continuous improvement of people's living standards, people's demand for beauty is also getting higher and higher. Along with this, the products of beauty flexible masks and beauty instruments are selling well, especially the beauty instruments. Because the beauty instruments can produce micro-current stimulation on the human face to make human get more beautiful, and the beauty instruments are loved by more and more people. The existing beauty instruments are all hand-held beauty instruments. When used, people need to operate it in front of a mirror. This makes the hand-held beauty instrument very inconvenient to use.

### BRIEF DESCRIPTION OF THE DRAWINGS

Implementations of the present technology will now be described, by way of example only, with reference to the attached figures, wherein:

FIG. 1 is a schematic view of a beauty instrument with mask according to a first embodiment.

FIG. 2 is a schematic view of a second flexible layer used in the beauty instrument with mask according to one embodiment.

FIG. 3 shows a Scanning Electron Microscope (SEM) image of a drawn carbon nanotube film.

FIG. 4 is a schematic view of carbon nanotube segments in the drawn carbon nanotube film.

FIG. 5 shows an SEM image of a flocculated carbon nanotube film.

FIG. 6 shows an SEM image of a pressed carbon nanotube film.

FIG. 7 shows a schematic view of a heating layer including a plurality of carbon nanotube wires crossed with each other.

FIG. 8 shows a schematic view of a heating layer including a plurality of carbon nanotube wires waved with each other.

FIG. 9 shows a schematic view of a heating layer including a bended and winded carbon nanotube wire.

FIG. 10 is an SEM image of an untwisted carbon nanotube wire.

FIG. 11 is an SEM image of twisted carbon nanotube wire.

FIG. 12 is a schematic view of a beauty instrument with mask according to a second embodiment.

FIG. 13 is a schematic view of part of a beauty instrument with mask according to a third embodiment.

### DETAILED DESCRIPTION

The disclosure is illustrated by way of example and not by way of limitation in the figures of the accompanying drawings in which like references indicate similar elements. It should be noted that references to “another,” “an,” or “one” embodiment in this disclosure are not necessarily to the same embodiment, and such references mean “at least one.”

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts have been exaggerated to better illustrate details and features of the present disclosure.

Several definitions that apply throughout this disclosure will now be presented.

The term “contact” is defined as a direct and physical contact. The term “substantially” is defined to be that while essentially conforming to the particular dimension, shape, or other feature that is described, the component is not or need not be exactly conforming to the description. The term “comprising,” when utilized, means “including, but not necessarily limited to”; it specifically indicates open-ended inclusion or membership in the so-described combination, group, series, and the like.

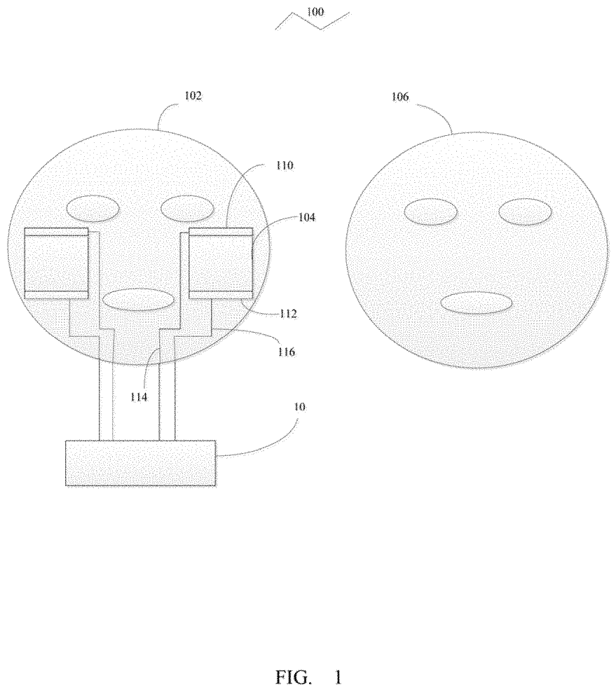

Referring to FIG. 1, a beauty instrument with mask according to a first embodiment is provided. The beauty instrument with mask includes a flexible mask 100 and a controller 10 for controlling the flexible mask 100. The flexible mask 100 includes a first flexible layer 102 and a second flexible layer 106 overlapped with each other (for clarity of display, in FIG. 1, the first flexible layer 102 and the second flexible layer 106 are separately shown), the first flexible layer 102 and the second flexible layer 106 have corresponding eye and mouth openings (not labeled); and at least a heating layer 104 located between the first flexible layer 102 and the second flexible layer 106; at least one first electrode 110 and at least one second electrode 112, each heating layer 104 is electrically connected with one first electrode 110 and one second electrode 112; at least one first electrode lead 114 and at least one second electrode lead 114, one first electrode 110 is electrically connected to one first electrode lead 114, and one second electrode 112 is electrically connected to one second electrode lead 116.

The at least one heating layer 104 can be a plurality of heating layers 104, or one heating layer 104. As can be shown in FIG. 1, the flexible mask 100 includes two heating layers 104. The two heating layers 104 are symmetrically distributed at a cheek position of a human face. When the flexible mask 100 includes a plurality of heating layers 104, the position of the heating layer 104 is not limited, and can be a forehead position, a cheek position, an eye below position, a nose position, or the like. The number of the heating layers 104 is not limited and can be adjusted as needed, and may be 2, 8, 15, 20 or the like. An area of each heating layer 104 is not limited and can be adjusted as needed. Adjacent heating layers 104 are spaced apart and insulated from each other.

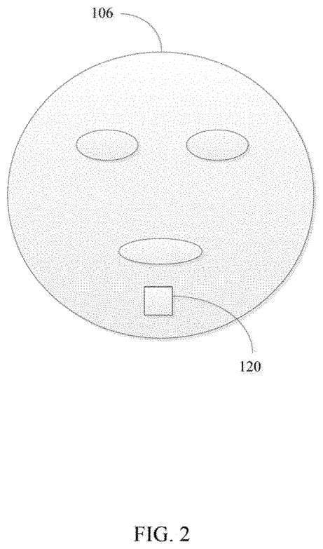

The controller 10includes a plurality of function buttons for controlling the flexible mask 100. The controller 10 is electrically connected to the flexible mask 100 through the at least one first electrode lead 114 and the at least one second electrode lead 116. Each function button can control the current magnitude, the frequency of the current, the position of the input current, etc., to control the heating layer 104 inside the flexible mask 100 to achieve the purpose of heating. The flexible mask 100 can be movably coupled to the controller 10. Optionally, the first flexible layer 102 or the second flexible layer 106 can include a window, and the first electrode lead 114 and the second electrode lead 116 are exposed from the window and electrically connected to the controller. Please referring to FIG. 2, in one embodiment, the second flexible layer 106 defines a window 120, In this embodiment, the flexible mask 100 includes a window 108 defined by the first flexible layer 102. The window 108 is provided with an access port through which the controller is connected to the flexible mask 100. The flexible mask 100 can be replaced as needed. The flexible mask 100 can also be cleaned for reuse.

A material of the first flexible layer 102 or the second flexible layer 104 can be a flexible material such as non-woven fabric, silk, flexible cloth, porous flexible paper, or silica gel, and can be directly attached to a person's face. A thickness of the first flexible layer 102 or the second flexible layer 104 can be set according to actual needs. In this embodiment, the thickness of the first flexible layer 102 or the second flexible layer 104 is in a range from 10 to 100 micrometers. The first flexible layer 102 or the second flexible layer 104 can be a porous structure or a non-porous structure.

In one embodiment, the heating layer 104 comprises a carbon nanotube layer or is the carbon nanotube layer. The carbon nanotube layer includes a plurality of carbon nanotubes joined by van der Waals attractive force therebetween. The carbon nanotube layer can be a substantially pure structure of carbon nanotubes, with few impurities. The carbon nanotube layer can be a freestanding structure, that is, the carbon nanotube layer can be supported by itself without a substrate. For example, if at least one point of the carbon nanotube layer is held, the entire carbon nanotube layer can be lifted while remaining its structural integrity.

The carbon nanotubes in the carbon nanotube layer can be orderly or disorderly arranged. The term ‘disordered carbon nanotube layer’ refers to a structure where the carbon nanotubes are arranged along different directions, and the aligning directions of the carbon nanotubes are random. The number of the carbon nanotubes arranged along each different direction can be almost the same (e.g. uniformly disordered). The disordered carbon nanotube layer can be isotropic, namely the carbon nanotube layer has properties identical in all directions of the carbon nanotube layer. The carbon nanotubes in the disordered carbon nanotube layer can be entangled with each other.

The carbon nanotube layer including ordered carbon nanotubes is an ordered carbon nanotube layer. The term ‘ordered carbon nanotube layer’ refers to a structure where the carbon nanotubes are arranged in a consistently systematic manner, e.g., the carbon nanotubes are arranged approximately along a same direction and/or have two or more sections within each of which the carbon nanotubes are arranged approximately along a same direction (different sections can have different directions). The carbon nanotubes in the carbon nanotube layer can be selected from single-walled, double-walled, and/or multi-walled carbon nanotubes. The carbon nanotube layer can include at least one carbon nanotube film. In other embodiments, the carbon nanotube layer is composed of one carbon nanotube film or at least two carbon nanotube films. In other embodiment, the carbon nanotube layer consists one carbon nanotube film or at least two carbon nanotube films.

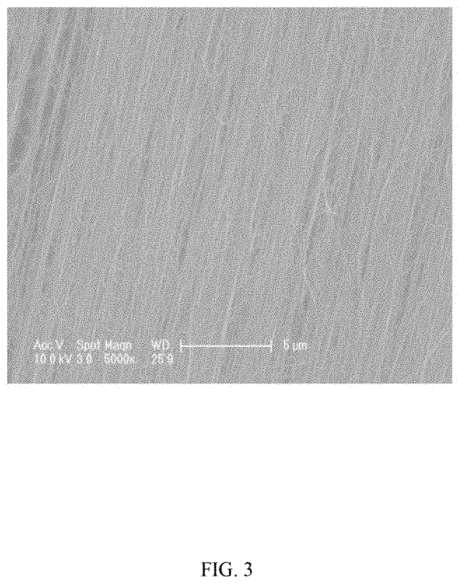

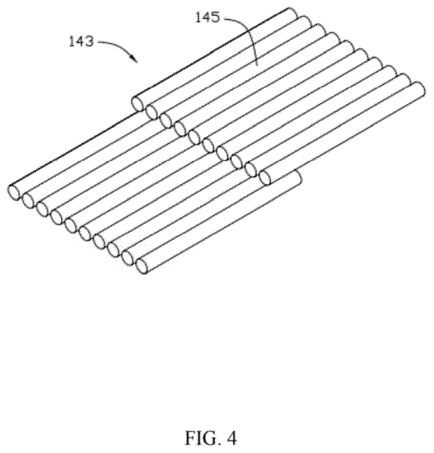

In one embodiment, the carbon nanotube film can be a drawn carbon nanotube film. Referring to FIG. 3, the drawn carbon nanotube film includes a number of successive and oriented carbon nanotubes joined end-to-end by van der Waals attractive force therebetween. The drawn carbon nanotube film is a freestanding film. Each drawn carbon nanotube film includes a number of successively oriented carbon nanotube segments joined end-to-end by van der Waals attractive force therebetween. Referring to FIG. 4, each carbon nanotube segment 143 includes a number of carbon nanotubes 145 substantially parallel to each other, and joined by van der Waals attractive force therebetween. Some variations can occur in the drawn carbon nanotube film. The carbon nanotubes in the drawn carbon nanotube film are oriented along a preferred orientation. The drawn carbon nanotube film can be treated with an organic solvent to increase the mechanical strength and toughness of the drawn carbon nanotube film and reduce the coefficient of friction of the drawn carbon nanotube film. The thickness of the drawn carbon nanotube film can range from about 0.5 nanometers to about 100 micrometers. The drawn carbon nanotube layer can be used as a carbon nanotube layer directly.

The carbon nanotubes in the drawn carbon nanotube film can be single-walled, double-walled, and/or multi-walled carbon nanotubes. The diameters of the single-walled carbon nanotubes can range from about 0.5 nanometers to about 50 nanometers. The diameters of the double-walled carbon nanotubes can range from about 1 nanometer to about 50 nanometers. The diameters of the multi-walled carbon nanotubes can range from about 1.5 nanometers to about 50 nanometers. The lengths of the carbon nanotubes can range from about 200 micrometers to about 900 micrometers.

The carbon nanotube layer can include at least two stacked drawn carbon nanotube films. The carbon nanotubes in the drawn carbon nanotube film are aligned along one preferred orientation, an angle can exist between the orientations of carbon nanotubes in adjacent drawn carbon nanotube films, whether stacked or adjacent. An angle between the aligned directions of the carbon nanotubes in two adjacent drawn carbon nanotube films can range from about 0 degrees to about 90 degrees (e.g. about 15 degrees, 45 degrees or 60 degrees).

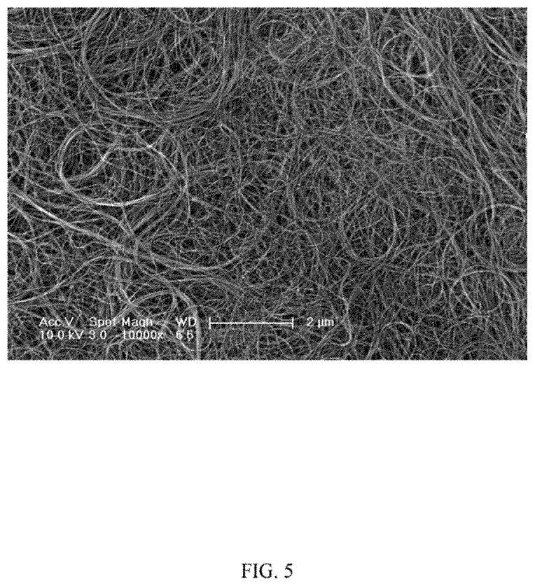

In other embodiments, the carbon nanotube film can be a flocculated carbon nanotube film. Referring to FIG. 5, the flocculated carbon nanotube film can include a plurality of long, curved, disordered carbon nanotubes entangled with each other.

Furthermore, the flocculated carbon nanotube film can be isotropic. The carbon nanotubes can be substantially uniformly dispersed in the carbon nanotube film. Adjacent carbon nanotubes are acted upon by van der Waals attractive force to obtain an entangled structure with micropores defined therein. Because the carbon nanotubes in the carbon nanotube layer are entangled with each other, the carbon nanotube layer employing the flocculated carbon nanotube film has excellent durability, and can be fashioned into desired shapes with a low risk to the integrity of the carbon nanotube layer. The thickness of the flocculated carbon nanotube film can range from about 0.5 nanometers to about 1 millimeter.

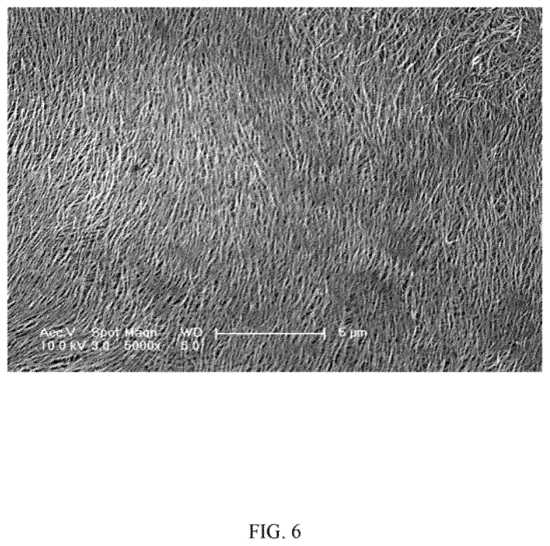

Referring to FIG. 6, in other embodiments, the carbon nanotube film can be a pressed carbon nanotube film. The pressed carbon nanotube film is formed by pressing a carbon nanotube array. The carbon nanotubes in the pressed carbon nanotube film are arranged along a same direction or along different directions. The carbon nanotubes in the pressed carbon nanotube film can rest upon each other. Adjacent carbon nanotubes are attracted to each other and are joined by van der Waals attractive force. An angle between a primary alignment direction of the carbon nanotubes and a surface of the pressed carbon nanotube film is about 0 degrees to approximately 15 degrees. The greater the pressure applied, the smaller the angle obtained. In one embodiment, the carbon nanotubes in the pressed carbon nanotube film are arranged along different directions, the carbon nanotube layer can be isotropic. The thickness of the pressed carbon nanotube film can range from about 0.5 nanometers to about 1 millimeter.

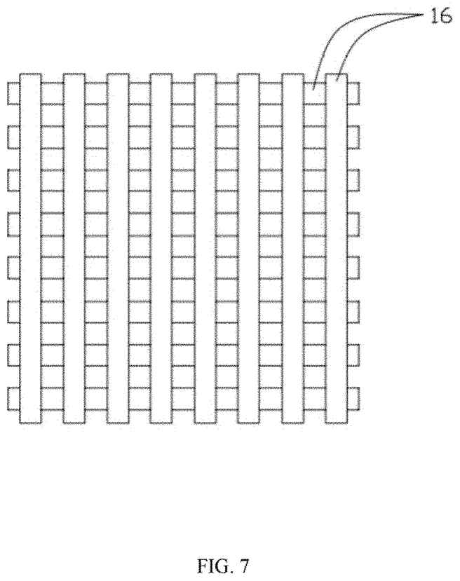

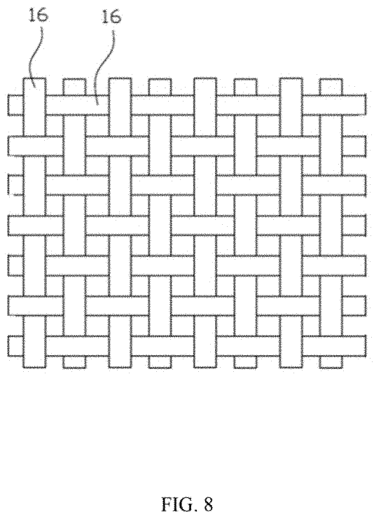

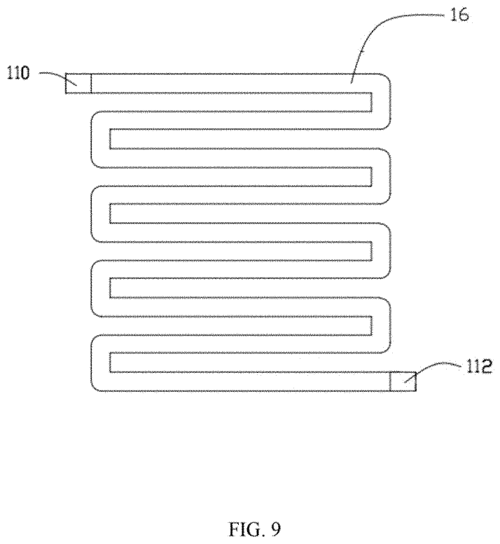

In some embodiments, the carbon nanotube layer can include a plurality of carbon nanotube wires. Referring to FIG. 7, the plurality of carbon nanotube wires 16 can be crossed with each other to form the carbon nanotube layer. Referring to FIG. 8, the plurality of carbon nanotube wires 16 can be waved with each other to form the carbon nanotube layer. In other embodiments, the carbon nanotube layer can include only one carbon nanotube wire. Referring to FIG. 9, the carbon nanotube wire 16 can be bended to form the carbon nanotube layer.

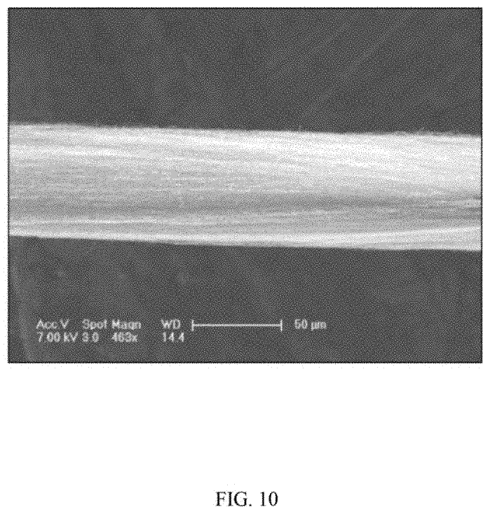

The carbon nanotube wire can be untwisted or twisted. Referring to FIG. 10, the untwisted carbon nanotube wire includes a plurality of carbon nanotubes substantially oriented along a same direction (i.e., a direction along the length direction of the untwisted carbon nanotube wire). The untwisted carbon nanotube wire can be a pure structure of carbon nanotubes. The untwisted carbon nanotube wire can be a freestanding structure. The carbon nanotubes are substantially parallel to the axis of the untwisted carbon nanotube wire. In one embodiment, the untwisted carbon nanotube wire includes a plurality of successive carbon nanotube segments joined end to end by van der Waals attractive force therebetween. Each carbon nanotube segment includes a plurality of carbon nanotubes substantially parallel to each other, and combined by van der Waals attractive force therebetween. The carbon nanotube segments can vary in width, thickness, uniformity and shape. Length of the untwisted carbon nanotube wire can be arbitrarily set as desired. A diameter of the untwisted carbon nanotube wire ranges from about 50 nanometers to about 100 micrometers.

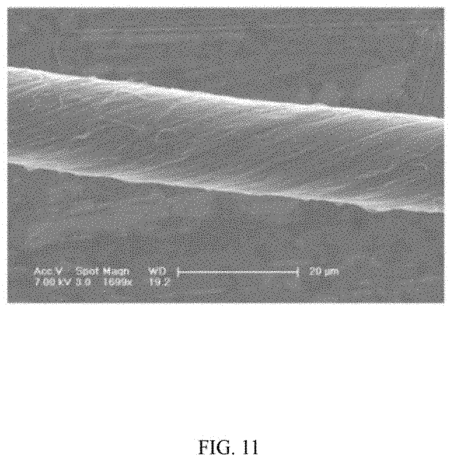

Referring to FIG. 11, the twisted carbon nanotube wire includes a plurality of carbon nanotubes helically oriented around an axial direction of the twisted carbon nanotube wire. The twisted carbon nanotube wire can be a pure structure of carbon nanotubes. The twisted carbon nanotube wire can be a freestanding structure. In one embodiment, the twisted carbon nanotube wire includes a plurality of successive carbon nanotube segments joined end to end by van der Waals attractive force therebetween.

Each carbon nanotube segment includes a plurality of carbon nanotubes substantially parallel to each other, and combined by van der Waals attractive force therebetween. The length of the carbon nanotube wire can be set as desired. A diameter of the twisted carbon nanotube wire can be from about 50 nanometers to about 100 micrometers. Furthermore, the twisted carbon nanotube wire can be treated with a volatile organic solvent after being twisted. After being soaked by the organic solvent, the adjacent substantially parallel carbon nanotubes in the twisted carbon nanotube wire will bundle together, due to the surface tension of the organic solvent when the organic solvent volatilizes. The density and strength of the twisted carbon nanotube wire will increase.

The carbon nanotube layer has a better flexibility than the first flexible layer and/or the second flexible layer. When the carbon nanotube layer is used as the heating layer in the flexible mask, the flexibility of the entire flexible mask is not decreased by the heating layer. The carbon nanotube layer has a large strength, as such, no matter how the flexible mask is bent or pulled, and the carbon nanotube layer is not damaged.

In other embodiments, each heating layer 104 can further include a graphene layer. That is, each heating layer 104 includes the carbon nanotube layer and the graphene layer overlapped with each other. The graphene layer includes at least one graphene. In one embodiment, the graphene layer is a pure structure of graphenes. The graphene layer structure can include a single graphene or a plurality of 1 graphenes. In one embodiment, the graphene layer includes a plurality of graphenes, the plurality of graphenes are stacked with each other and/or located side by side. The plurality of graphenes is combined with each other by van der Waals attractive force. The graphene layer can be a continuous integrated structure. The term “continuous integrated structure” can be defined as a structure that is combined by a plurality of chemical covalent bonds (e.g., sp² bonds, sp¹ bonds, or sp³ bonds) to form an overall structure. A thickness of the graphene layer can be less than 1 millimeter.

Reference to FIG. 1 again, each heating layer 104 is electrically coupled with a first electrode 110 and a second electrode 112. The first electrode 110 and the second electrode 112 are separately located at both ends of the heating layer 104 and are located on a surface of the heating layer 104. The first electrode 110 and the second electrode 112 are directly located the surface of the heating layer 104. In use, a voltage is applied between the first electrode 110 and the second electrode 112, and a current flows inside the heating layer 104 to generate heat. The voltage between the first electrode 110 and the second electrode 112 can be controlled by the controller 10, and a temperature of the heating layer 104 is controlled. By adjusting the controller 10, it is also possible to selectively control which heating layer 104 is heated to selectively heat the face region.

The first electrode 110 and the second electrode 112 can be a conductive film, a metal piece or a metal lead. Preferably, each of the first electrode 110 and the second electrode 112 is a linear conductive film, and a thickness of the linear conductive film is not limited. A material of the first electrode 110 and the second electrode 112 can be metal, alloy, indium tin oxide (ITO), antimony tin oxide (ATO), conductive silver paste, conductive polymer or conductive carbon nanotube. The metal or the alloy can be aluminum, copper, tungsten, molybdenum, gold, titanium, rhodium, palladium, iridium or any alloy thereof. In this embodiment, the first electrode 110 and the second electrode 112 are linear copper conductive films having the thickness of 1 micrometer. The first electrode 110 and the second electrode 112 should have better flexibility and a smaller thickness. Preferably, an insulating layer (not shown) can be located on the surfaces of the first electrode 110 and the second electrode 112 to prevent the first electrode 110 and the second electrode 112 from being oxidized when in use.

A material of the first electrode lead 114 or the second electrode lead 116 can be metal, alloy, indium tin oxide (ITO), antimony tin oxide (ATO), conductive silver paste, conductive polymer or conductive carbon nanotube. The metal or the alloy can be aluminum, copper, tungsten, molybdenum, gold, titanium, rhodium, palladium, iridium or any alloy thereof. In this embodiment, the first electrode lead 114 and the second electrode lead 116 are both copper wires. Preferably, an insulating layer can be coated on the surface of each of the first electrode lead 114 or the second electrode lead 116. The material of the insulating layer is a flexible material.

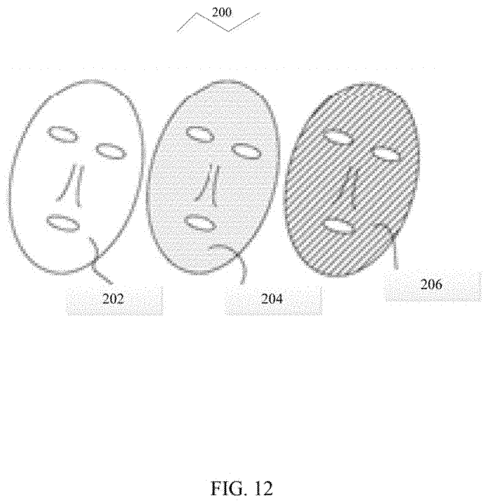

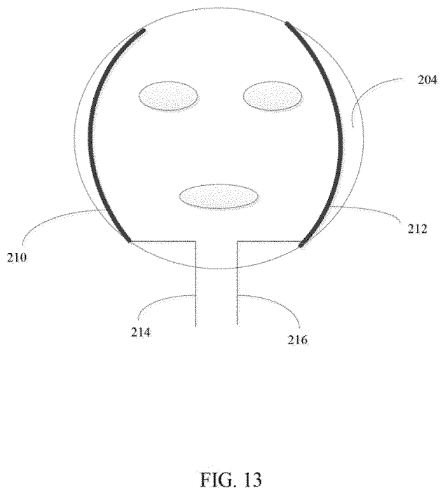

A beauty instrument with mask according to a second embodiment is provided. The beauty instrument with mask comprises a flexible mask and a controller. Referring to FIG. 12 and FIG. 13, the flexible mask 200 includes a first flexible layer 202, a second flexible layer 206, the first flexible layer 202 and the second flexible layer 206 are stacked with each other; at least one heating layer 204 located between the first flexible layer 202 and the second flexible layer 206. In this embodiment, only one heating layer 204 located between the first flexible layer 202 and the second flexible layer 206. The heating layer 204 has corresponding openings for the eyes and mouth. The first electrode 110 and the second electrode 112 are respectively located at two ends of the heating layer 204, and are arc-shaped conductive films that match the heating layer 204. The first electrode 110 and the second electrode 112 are electrically connected to the controller (not shown) through a first electrode lead (not shown) and a second electrode lead (not shown). The beauty instrument with mask provided in this embodiment can realize full face heating when working.

Other characteristics of the beauty instrument with mask in the second embodiment are the same as that of the beauty instrument with mask in the first embodiment.

The present invention further provides a method of using the beauty instrument with mask, the method comprises the steps of:
Step S1: providing the beauty instrument with mask;
Step S2: applying the flexible mask of the beauty instrument with mask on a user's face; and
Step S3: turning on the controller and selecting a function button on the controller, inputting a current to the at least one heating layer in the flexible mask, and heating the at least one heating layer.

In the step S1, the beauty instrument with mask is any one of the beauty instrument with masks discussed above.

Alternatively, before step S2, the flexible mask can be further infiltrated with a liquid, that is, before the flexible mask of the beauty instrument with mask is applied on the user's face. The liquid can be a cosmetic liquid. After the flexible mask is heated, it can promote the absorption of the beauty liquid and achieve a cosmetic effect on the user's face.

In step S3, the controller includes a plurality of function buttons for controlling the flexible mask. Each function button is used to control the heating layer inside the flexible mask to achieve the heating function. Each function button can be configured to control the a current magnitude, a current frequency, a position of the heating layer which the current is input. The controller can control the heating layer inside the flexible mask to simultaneously heat, or selectively control a certain heating layer or some certain heating layers. For example, when the heating layers are located at a forehead position, a cheek position and a chin position, the controller can control the heating layers in these positions circulation heat in the order of the forehead position, the cheek position, and the chin position.

The flexible mask can be movably coupled to the controller. The flexible mask defines an access at the window position on the first flexible layer or the second flexible layer, and the controller is connected to the flexible mask through the access. The flexible mask can be changed as needed. The flexible mask can also be cleaned to achieve re-use purpose.

Compared with the prior art, the beauty instrument with mask provided by the present invention has the following advantages: first, it can directly fit on a user's face without the need to hold it by hand, which frees the user's hands. Secondly, through controlling a circuit by the controller, the skin on the user's face can be selectively stimulated, and the face parts to be stimulated can be selected more accurately without causing facial asymmetry. Third, the carbon nanotube layer is used as the heating layer, the carbon nanotube layer has a better flexibility than the first flexible layer or/and the second flexible layer, and the flexibility of the entire flexible mask will not be reduced due to the setting of the heating layers, the flexible mask can fit on the user's face well, and the user has a high comfort degree. Fourth, the carbon nanotube layer is used as a heating layer, a strength of the carbon nanotube layer is relatively large, no matter how to bend and pull or clean the flexible mask, the carbon nanotube layer will not be damaged, and the flexible mask has a long life.

Depending on the embodiment, certain blocks/steps of the methods described may be removed, others may be added, and the sequence of blocks may be altered. It is also to be understood that the description and the claims drawn to a method may comprise some indication in reference to certain blocks/steps. However, the indication used is only to be viewed for identification purposes and not as a suggestion as to an order for the blocks/steps.

The embodiments shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size, and arrangement of the parts within the principles of the present disclosure, up to and including the full extent established by the broad general meaning of the terms used in the claims. It will therefore be appreciated that the embodiments described above may be modified within the scope of the claims.

What is claimed is:

1. A method for using beauty instrument with mask, comprising:
Step S1: providing a beauty instrument with mask, the beauty instrument with mask comprises a flexible mask and a controller comprising a plurality of a plurality of function buttons, wherein the flexible mask comprises:
a first flexible layer;
a second flexible layer overlapped with the first flexible layer;
at least one heating layer sandwiched between the first flexible layer and the second flexible layer, wherein the at least one heating layer comprises a carbon nanotube layer, the carbon nanotube layer comprises a plurality of carbon nanotubes uniformly distributed;
at least one first electrode and at least one second electrode, each of the at least one first electrode and the at least one second electrode are electrically connected with the at least one heating layer;
at least one first electrode lead electrically coupled with the at least one first electrode and at least one second electrode electrically connected with and at least one second electrode, wherein the flexible mask is electrically coupled with the controller from the at least one first electrode lead and the at least one second electrode lead;
Step S2: applying the flexible mask of the beauty instrument with mask on a face; and
Step S3: turning on the controller and selecting a function button from the plurality of function buttons on the controller, inputting a current to the at least one heating layer in the flexible mask, and heating the at least one heating layer.

2. The method of claim 1, wherein the first flexible layer or the second flexible layer defines a window, and each of the at least one first electrode lead and the at least one second electrode lead is exposed from the window and electrically connected to the controller.

3. The method of claim 1, wherein the flexible mask is movably coupled to the controller.

4. The method of claim 1, wherein before step S2, the flexible mask is infiltrated with a liquid.

5. The method of claim 1, wherein the plurality of function buttons are configured to control a current magnitude, a current frequency, a position of the heating layer which the current is input.

6. The method of claim 1, wherein the at least one heating layer comprises one heating layer or a plurality of heating layers.

7. The method of claim 6, wherein the at least one heating layer comprises a plurality of heating layers located at a forehead position, a cheek position or a chin position.

8. The method of claim 1, wherein the carbon nanotube layer comprises one carbon nanotube film or a plurality of carbon nanotube films overlapped with each other.

9. The method of claim 8, wherein the carbon nanotube film comprises a plurality of successive and oriented carbon nanotubes joined end-to-end by van der Waals attractive force therebetween.

10. The method of claim 9, wherein the carbon nanotube film comprises a plurality of successively oriented carbon nanotube segments joined end-to-end by van der Waals attractive force therebetween, and each carbon nanotube segment comprises a plurality of carbon nanotubes substantially parallel to each other, and joined by van der Waals attractive force therebetween.

11. The method of claim 8, wherein the carbon nanotube film comprises a plurality of carbon nanotubes entangled with each other.

12. The method of claim 8, wherein the carbon nanotube film comprises a plurality of carbon nanotubes joined by van der Waals attractive force, an angle between a primary alignment direction of the carbon nanotubes and a surface of the carbon nanotube film is ranged from 0 degrees to 15 degrees.

13. The method of claim 1, wherein the carbon nanotube layer comprises at least one carbon nanotube wire, the at least one carbon nanotube wire comprises a plurality of successive carbon nanotube segments joined end to end by van der Waals attractive force therebetween and oriented along a length direction of the at least one carbon nanotube wire.

14. The method of claim 13, wherein the carbon nanotube layer comprises one carbon nanotube wire, the carbon nanotube wire is bended to form the carbon nanotube layer.

15. The method of claim 13, wherein the carbon nanotube layer comprises a plurality of carbon nanotube wires crossed or weaved with each other.

16. The method of claim 1, wherein a material of the at least one first electrode or the at least one second electrode is metal, alloy, indium tin oxide (ITO), antimony tin oxide (ATO), conductive silver paste, conductive polymer or conductive carbon nanotube.

17. The method of claim 1, wherein the at least one heating layer is one heating layer, the at least one first electrode is one first electrode, the at least one second electrode is one second electrode, the first electrode and the second electrode are respectively located at two ends of the heating layer.

18. The method of claim 17, wherein the first electrode and the second electrode are arc-shaped conductive films matching a profile of the heating layer.

19. The method of claim 1, wherein the at least one heating layer comprises a graphene layer overlapped with the carbon nanotube layer.

20. The method of claim 19, wherein the graphene layer comprises a plurality of graphenes, the plurality of graphenes are stacked with each other or located side by side.
